(19) United States
(12) Patent Application Publication
(10) Pub. No.: US 2004/0242650 A1
(43) Pub. Date: Dec. 2, 2004

(54) Extended dosing regimen

(76) Inventor: Anne E. Hagey, Lake Forest, IL (US)

Correspondence Address: ROBERT DEBERARDINE, ABBOTT LABORATORIES, 100 ABBOTT PARK ROAD, DEPT. 377/AP6A, ABBOTT PARK, IL, 60064-6008, US

(21) Appl. No.: 10/842,667

(22) Filed: May 10, 2004

Related U.S. Application Data
(63) Continuation-in-part of application No. 10/447,588, filed on May 29, 2003.

(51) Int. Cl. A61K 31/44

(52) U.S. Cl. 514/352

(57) ABSTRACT

The present invention relates to an extended dosing regimen of tubulin binding agents. Also disclosed are methods of treating diseases by dosing tubulin binding agents for extended periods of time.

This application is a continuation-in-part of U.S. application Ser. No. 10/447,558, filed May 29, 2003, the specification of which is hereby incorporated by reference.

### TECHNICAL FIELD

The present invention relates to an extended dosing regimen of tubulin binding agents. Also disclosed are methods of treating diseases by dosing tubulin binding agents for extended periods of time.

### BACKGROUND OF THE INVENTION

Tubulin is the protein that polymerizes into long chains or filaments that form microtubules, hollow fibers that serve as a skeletal system for living cells.

Microtubules have the ability to shift through various formations which is what enables a cell to undergo mitosis or to regulate intracellular transport. The formation-shifting of microtubules is made possible by the flexibility of tubulin which is why scientists have sought to understand the protein's atomic structure since its discovery in the 1950s. Certain anticancer drugs bind to tubulin and cause the protein to lose its flexibility, preventing the cell from dividing.

Approved tubulin binding agents consist of the taxanes (including paclitaxel and docetaxel) and the vinca alkaloids (comprised of three agents, vincristine, vinblastine, and vinorelbine). Typically these agents are administered intraveneously and are dosed every one to three weeks due to the adverse reactions suffered by patients, including neurotoxicity, neutropenia, hypersensitivity, and other harmful side effects. Thus, there is a continuing need for a dosing regimen that allows tubulin binding agents to be administered for longer periods of time to maximize their anti-cancer effect.

### SUMMARY OF THE INVENTION

In its principle embodiment the present invention discloses a method of administering an oral tubulin binding agent, the method comprising administering the oral tubulin binding agent at least once per day over an extended period of time. In a preferred embodiment the oral tubulin binding agent binds to the colchicine binding site. In a more preferred embodiment the oral tubulin binding agent is N-[2-[(4-hydroxyphenyl)amino]-3-pyridyl]-4-methoxybenzenesulfonamide.

In another preferred embodiment the the oral tubulin binding agent is administered once or twice per day.

In another preferred embodiment the oral tubulin binding agent is administered in an amount between about 25 mg and about 500 mg per day. More preferably the oral tubulin binding agent is administered in an amount between about 25 mg and about 200 mg per day.

In another preferred embodiment the extended period of time is between about 7 and about 28 days. In a more preferred embodiment the extended period of time is about 7 days. In another more preferred embodiment the extended period of time is about 14 days. In another more preferred embodiment the extended period of time is about 21 days. In another more preferred embodiment the extended period of time is about 28 days.

In another embodiment the present invention method of treating a disease, the method comprising administering an oral tubulin binding agent at least once per day over an extended period of time. In a preferred embodiment the disease is cancer. In a more preferred embodiment the cancer is selected from the group consisting of leukemia, neuroblastoma, cervical, colorectal, renal, and melonoma. In a most preferred embodiment the cancer is colorectal.

### DETAILED DESCRIPTION OF THE INVENTION

All publications, issued patents, and patent applications cited herein are hereby incorporated by reference.

As used in the present specification the following terms have the meanings indicated:

The term “extended period of time,” as used herein, refers to an amount of time in excess of five days. Preferably, the extended period of time is a multiple of 7 days (i.e., 7, 14, 21, or 28 days).

The term “oral tubulin binding agent,” as used herein, refers to an orally dosed drug which is useful in the treatment of disorders mediated by tubulin. Examples of tubulin binding agents include paclitaxel, N-[2-[(4-hydroxyphenyl)amino]-3-pyridyl]-4-methoxybenzenesulfonamide, E7070, combretastatin A4 phosphate, the epothilones, docetaxel, taxotere, vincristine, vinblastine, and vinorelbine. Most preferably the oral tubuling binding agent is N-[2-[(4-hydroxyphenyl)amino]-3-pyridyl]-4-methoxybenzenesulfonamide.

The compounds of the invention may be useful in the treatment of diseases when used alone or in combination with other therapies. For example, when used for the treatment of cancer, the compounds of the invention may be administered alone or in combination with radiotherapy, hormonal agents, antibodies, antiangiogenics, COX-2 inhibitors, and/or other chemotherapeutic agents (cytotoxic and/or cytostatic) such as cisplatin, 5-fluorouracil, taxotere, and gemcitabine.

The compounds of the present invention may be used in the treatment of diseases mediated by tubulin. Such diseases include cancers such as neuroblastoma, cervical, renal, melonoma, breast (ductal and lobular), colorectal, lung (small cell and non-small cell), prostate, pancreatic, sarcoma, leukemia, lymphoma, and other bone marrow dyscrasias.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

N-[2-[(4-hydroxyphenyl)amino]-3-pyridyl]-4-methoxybenzenesulfonamide was prepared following the procedure described in U.S. Pat. No. 5,292,758, issued Mar. 8, 1994, which is hereby incorporated by reference in its entirety.

### EXAMPLE 1

### Preparation of Capsules of N-[2-[(4-hydroxyphenyl)amino]-3-pyridyl]-4-methoxybenzenesulfonamide

TABLE 1
Formulation of N-[2-[(4-hydroxyphenyl)amino]-3-
pyridyl]-4-methoxybenzenesulfonamide

| Ingredient | % w/w | Purpose |
|---|---|---|
| N-[2-[(4- | 30.0 | |
| hydroxyphenyl)amino]-3- | | |
| pyridyl]-4- | | |
| methoxybenzenesulfonamide | | |
| cellulose, microcrystalline, | 15.8 | Filler |
| NF (Avicel ® PH101) | | |
| lactose (monohydrate) | 28.0 | Filler |
| povidone, USP, K29-32 | 8.0 | Binder |
| croscarmellose Na | 18.0 | Disintegrant |
| water | sufficient quantity | Binder Liquid |
| magnesium stearate | 0.2 | Lubricant |

The povidone was dissolved in water. The Avicel®, N-[2-[(4-hydroxyphenyl)amino]-3-pyridyl]-4-methoxybenzenesulfonamide, lactose, and croscarmellose were mixed together. The mixture was granulated with the povidone solution and the resulting granulation was dried and then milled. The milled product was blended with magnesium stearate. The 25 mg and 100 mg doses were prepared by filling capsules with the appropriate weight of blended product. The 50 mg, 75 mg, 150 mg, and 200 mg doses were accomplished by combining the appropriate combinations of 25 mg and/or 100 mg capsules.

### EXAMPLE 2

### Evaluation of Extended Dosing Regimen of N-[2-[(4-hydroxyphenyl)amino]-3-pyridyl]-4-methoxybenzenesulfonamide

A total of 43 patients were enrolled in the study. The tumor types were as follows: colorectal (23), sarcoma (5), mesothelioma (3), salivary gland (2), endometrial (2), unknown (2), hepatoma (1), melanoma (1), renal cell (1), lung (1), ovary (1), and granulosa cell (1). Patients were treated once a day (QD) or twice a day (BID) for 21 days with N-[2-[(4-hydroxyphenyl)amino]-3-pyridyl]-4-methoxybenzenesulfonamide followed by a 7-day period where no drug was received. Doses were escalated by 50 mg/day (25 mg BID). Three patients were initially treated at each dose level. If dose-limiting toxicity (defined below) was observed in cycle one, three more patients were added to that dosing regimen. If additional patients experienced dose-limiting toxicity, on occasion the dose level was expanded to nine patients to further assess tolerability. Response assessment was performed every two cycles.

Dose limiting toxicities that were observed included ileus, peripheral neuropathy, fatigue, and abdominal pain. No dose limiting toxicity was seen in patients receiving up to 150 mg of drug per day.

In evaluating the pharmacokinetic profiles of the patients, plasma samples were collected pre-dose and over 6 hours following dosing on day 15. Plasma concentrations of N-[2-[(4-hydroxyphenyl)amino]-3-pyridyl]-4-methoxybenzenesulfonamide were determined by a validated LCMS/MS assay. Pharmacokinetic parameter estimates were obtained using noncompartmental methods, and included maximum observed concentration (Cₘₐₓ), time to Cₘₐₓ (Tₘₐₓ), minimum observed concentration (Cₘᵢₙ), half-life (t_{1/2}), and area under the plasma concentration-time profile over a dosing interval (AUC_{τ}). To facilitate calculation of day 15 AUCs, pre-dose concentrations at the beginning and end of the dosing interval were assumed to be equal. Results are summarized below (Table 2).

TABLE 2
Pharmacokinetic Results

| | | | Tₘₐₓ | | t_{1/2}# | AUC_{τ}* |
|---|---|---|---|---|---|---|
| Regimen | N | Cₘₐₓ (mcg/mL) | (h) | Cₘᵢₙ (mcg/mL) | (h) | (h · mcg/mL) |
| 25 mg QD | 4 | 1.7 ± 0.3 | 1.3 ± 0.5 | 0.1 ± 0.0 | 6.3 ± 0.2 | 9.0 ± 2.9 |
| 50 mg QD | 3 | 3.5 ± 0.2 | 1.7 ± 0.6 | 0.1 ± 0.1 | 4.6 ± 2.4 | 20.4 ± 5.0 |
| 100 mg QD | 3 | 8.4 ± 0.9 | 1.7 ± 0.6 | 0.2 ± 0.1 | 5.8 ± 1.3 | 39.5 ± 7.9 |
| 150 mg QD | 3 | 8.2 ± 2.3 | 1.2 ± 0.8 | 0.7 ± 0.4 | 8.5 ± 1.5 | 55.1 ± 12.2 |
| 200 mg QD | 2 | 9.1 ± 5.9 | 2.0 ± 0.0 | 0.5 ± 0.3 | 7.0 ± 1.0 | 60.2 ± 34.2 |
| 25 mg BID | 2 | 2.0 ± 0.6 | 2.3 ± 2.5 | 0.3 ± 0.0 | 3.8 ± 1.7 | 9.1 ± 0.4 |
| 50 mg BID | 2 | 5.0 ± 0.9 | 0.5 ± 0.0 | 0.4 ± 0.2 | 4.0 ± 0.6 | 16.4 ± 3.2 |
| 75 mg BID | 3 | 3.7 ± 1.2 | 1.5 ± 0.9 | 0.6 ± 0.0 | 5.3 ± 0.9 | 17.0 ± 3.6 |
| 100 mg BID | 3 | 3.5 ± 1.3 | 2.3 ± 1.5 | 0.9 ± 0.4 | 6.1 ± 2.3 | 24.1 ± 10.0 |

#Summary statistics for t_{1/2} presented as harmonic mean and pseudo standard deviation.
*AUC₀₋₂₄ for QD regimens; AUC₀₋₁₂ for BID regimens.

Following oral dosing, N-[2-[(4-hydroxyphenyl)amino]-3-pyridyl]-4-methoxybenzenesulfonamide was rapidly absorbed; the overall mean Tₘₐₓ was 1.5 hours. After peaking, plasma concentrations declined with an overall mean t_{1/2} of 6 hours. As expected, for a given daily dose, Cₘᵢₙ concentrations tended to be greater for BID regimens compared to QD regimens. N-[2-[(4-hydroxyphenyl)amino]-3-pyridyl]-4-methoxybenzenesulfonamide concentrations increased proportionally with increasing dose, indicating dose-proportional (linear) pharmacokinetics across the range of doses studied. Tₘₐₓ and t_{1/2} did not appear to vary with dose, a finding that is also consistent with dose-proportional (linear) pharmacokinetics. Plasma concentrations of N-[2-[(4-hydroxyphenyl)amino]-3-pyridyl]-4-methoxybenzenesulfonamide accumulated minimally with QD or BID dosing.

Efficacy of N-[2-[(4-hydroxyphenyl)amino]-3-pyridyl]-4-methoxybenzenesulfonamide was tested in Calu-6 NSCL, MDA-MB-468 breast and HT-29 colon carcinoma xenografts grown in nude mice. The objectives were determination of the efficacy of N-[2-[(4-hydroxyphenyl)amino]-3-pyridyl]-4-methoxybenzenesulfonamide as a single agent and in combination with cisplatin in the Calu-6 NSCL flank xenograft model; determination the efficacy of N-[2-[(4-hydroxyphenyl)amino]-3-pyridyl]-4-methoxybenzenesulfonamide as a single agent and in combination with docetaxel in the MDA-MB 468 breast flank xenograft model; determination of the efficacy of N-[2-[(4-hydroxyphenyl)amino]-3-pyridyl]-4-methoxybenzenesulfonamide as a single agent and in combination with 5-FU in the HT-29 colon flank xenograft model; and comparison the efficacy of N-[2-[(4-hydroxyphenyl)amino]-3-pyridyl]-4-methoxybenzenesulfonamide administered on 2 different schedules (q.d. vs b.i.d.) in the Calu-6 and HT-29 xenogaft model.

Following inoculation of tumor cells, tumors were passaged three times in mice before using the fragments to generate single cell suspensions. Multiple animals were used as donors. Single cell suspensions were generated by homogenizing the minced tumor tissue in RPMI 1640 media. Cell suspensions were washed three times to remove debris and other foreign materials. Viable cells were counted by trypan blue exclusion technique using a hemocytometer. Mice were injected subcutaneously with 0.5 ml of the cell suspension containing 1×10⁶ cells.

The doses for N-[2-[(4-hydroxyphenyl)amino]-3-pyridyl]-4-methoxybenzenesulfonamide for oral dosing were determined by the MTD study performed on Jun. 17, 2002 in-house. For continuous 21 day q.d. dosing, 50 mg/kg/day was selected since this dose did not produce any toxicities. Higher doses must be cycled, 5 days on, 5 days off, for 2 cycles to determine efficacy without adverse toxicity.

Efficacy of N-[2-[(4-hydroxyphenyl)amino]-3-pyridyl]-4-methoxybenzenesulfonamide in the Calu-6 Flank Xenograft Model

Tumor cells were inoculated s.c. into male nude mice on day 0. On day 10, mice bearing established tumors were size matched at ˜233 mm³ and placed into the following groups:

N-[2-[(4-hydroxyphenyl)amino]-3-pyridyl]-4-methoxybenzenesulfonamide—100, and 75 mg/kg/day (p.o., q.d., 5 days on, 5 days off ×2)

N-[2-[(4-hydroxyphenyl)amino]-3-pyridyl]-4-methoxybenzenesulfonamide—50 and 0 mg/kg/day (p.o., q.d. ×21 or p.o., b.i.d. ×21)

Cisplaitn—20, 10, 8 and 0 mg/kg/day (ip, qd ×1)

Combination Therapy—100/10, 75/10, 100/8 mg/kg/day (p.o., q.d., 5 days on, 5 days off ×2/ip, qd ×1), 50/10 mg/kg/day (p.o., q.d., ×21/ ip, qd ×1)

In the Calu-6 xenograft model, N-[2-[(4-hydroxyphenyl)amino]-3-pyridyl]-4-methoxybenzenesulfonamide administered as a single agent at 100 and 75 mg/kg/day on a (5 days on, 5 days off for 2 cycles) schedule demonstrated significant antitumor activity. N-[2-[(4-hydroxyphenyl)amino]-3-pyridyl]-4-methoxybenzenesulfonamide at 50 mg/kg/day administered either on a q.d., or b.i.d., schedule demonstrated similar efficacy. In combination with cisplatin, N-[2-[(4-hydroxyphenyl)amino]-3-pyridyl]-4-methoxybenzenesulfonamide demonstrated greater than additive responses with all three doses of N-[2-[(4-hydroxyphenyl)amino]-3-pyridyl]-4-methoxybenzene sulfonamide tested.

TABLE 1
In vivo efficacy of N-[2-[(4-hydroxyphenyl)amino]-3-pyridyl]-4-
methoxybenzenesulfonamide alone and in combination with cisplatin in the Calu-6 lung
subcutaneous flank xenografts grown in male nude mice. Tumors were size
matched at ˜233 mm³ and therapy was initiated.

| | | | | % | |
|---|---|---|---|---|---|
| | | | Tumor | T/C^{b} | |
| | Dose | Route | Volume^{a} | Day | |
| Compound | (mg/kg/day) | Schedule | Day 38 | 38 | % ILS^{c} |
| N-[2-[(4- | 100 | p.o., q.d., (5 days on, | 1022 ± 48 | 37*** | 65*** |
| hydroxyphenyl)amino]- | | 5 days off) x2 | | | |
| 3- | | | | | |
| pyridyl]-4- | | | | | |
| methoxybenzenesulfonamide | | | | | |
| | 75 | | 1406 ± 65 | 50*** | 58*** |
| | 0 | | 2799 ± 111 | | |
| N-[2-[(4- | 50 | p.o., b.i.d x21 | 1534 ± 47 | 56*** | 58*** |
| hydroxyphenyl)amino]- | | | | | |
| 3- | | | | | |
| pyridyl]-4- | | | | | |
| methoxybenzenesulfonamide | | | | | |
| Vehicle (N-[2- | 0 | | 2728 ± 66 | | |
| [(4- | | | | | |
| hydroxyphenyl)amino]- | | | | | |
| 3- | | | | | |
| pyridyl]-4- | | | | | |
| methoxybenzenesulfonamide) | | | | | |
| N-[2-[(4- | 50 | p.o., q.d x21 | 1455 ± 65 | 54*** | 58*** |
| hydroxyphenyl)amino]- | | | | | |
| 3- | | | | | |
| pyridyl]-4- | | | | | |
| methoxybenzenesulfonamide | | | | | |
| Vehicle (N-[2- | 0 | | 2713 ± 77 | | |
| [(4- | | | | | |
| hydroxyphenyl)amino]- | | | | | |
| 3- | | | | | |
| pyridyl]-4- | | | | | |
| methoxybenzenesulfonamide) | | | | | |
| Cisplatin | 20 | i.p., q.d x1 | 1472 ± 75 | 56*** | 58*** |
| | 10 | | 914 ± 49 | 35*** | 71*** |
| | 8 | | 1649 ± 67 | 63*** | 29*** |
| Vehicle (Cis) | 0 | | 2624 ± 89 | | |
| N-[2-[(4- | 100/10 | p.o., q.d., (5 days on, 5 | 169 ± 14 | 6*** | 188***^{eg} |
| hydroxyphenyl)amino]- | | | | | |
| 3- | | | | | |
| pyridyl]-4- | | | | | |
| methoxybenzenesulfonamide/ | | | | | |
| Cis | | | | | |
| | 75/10 | days off) x2/i.p., q.d | 239 ± 22 | 9*** | 158***^{eh} |
| | | x1 | | | |
| | 100/8 | | 478 ± 24 | 17*** | 100***^{fg} |
| Vehicle (N-[2- | 0/0 | | 2759 ± 71 | | |
| [(4- | | | | | |
| hydroxyphenyl)amino]- | | | | | |
| 3- | | | | | |
| pyridyl]-4- | | | | | |
| methoxybenzenesulfonamide/ | | | | | |
| Cis) | | | | | |
| N-[2-[(4- | 50/10 | p.o., q.d.x21/i.p., q.d | 244 ± 20 | 9*** | 123*** |
| hydroxyphenyl)amino]- | | x1 | | | |
| 3- | | | | | |
| pyridyl]-4- | | | | | |
| methoxybenzenesulfonamide/ | | | | | |
| Cis | | | | | |
| Vehicle (N-[2- | | | 2699 ± 93 | | |
| [(4- | | | | | |
| hydroxyphenyl)amino]- | | | | | |
| 3- | | | | | |
| pyridyl]-4- | | | | | |
| methoxybenzenesulfonamide/ | | | | | |
| Cis) | | | | | |

^{a}Mean ± SEM
^{b}Ratio of tumor volume for treated vs. control (vehicle)
^{c}% Median increase in % ILS compared to vehicle treated controls in time to 1 cc
^{d}p values vs. vehicle, * <0.05, ** <0.01, *** <0.001
^{e}p < 0.001 compared to cisplatin 10 mkd, ^{f} p < 0.001 compared to cisplatin 8 mkd
^{g}p < 0.001 compared to N-[2-[(4-hydroxyphenyl)amino]-3-pyridyl]-4-methoxybenzenesulfonamide 100 mkd, ^{h} p < 0.001 compared to N-[2-[(4-hydroxyphenyl)amino]-3-pyridyl]-4-methoxybenzenesulfonamide, 75 mkd

Efficacy of N-[2-[(4-hydroxyphenyl)amino]-3-pyridyl]-4-methoxybenzenesulfonamide in the MDA-MB-468 Breast Flank Xenograft Model. Tumor cells derived from serially passaged tumor fragments were inoculated s.c. in female nude mice on day 0. On day 10, mice bearing established tumors were size matched at ˜231 mm³ and placed into the following groups:

N-[2-[(4-hydroxyphenyl)amino]-3-pyridyl]-4-methoxybenzenesulfonamide—100, and 75 mg/kg/day (p.o., q.d., 5 days on, 5 days off ×2)

N-[2-[(4-hydroxyphenyl)amino]-3-pyridyl]-4-methoxybenzenesulfonamide—50 and 0 mg/kg/day (p.o., q.d. ×21)

Docetaxel—40, 33.3, 16.7 and 0 mg/kg/day (i.v., qd ×1)

Combination Therapy—100/33.3, 75/33.3, 100/16.7mg/kg/day (p.o., q.d., 5 days on, 5 days off ×2/ iv, q.d)—50/33.3, 50/16.7 mg/kg/day (p.o., q.d., ×21/ip, qd ×1)

As a single agent, N-[2-[(4-hydroxyphenyl)amino]-3-pyridyl]-4-methoxybenzenesulfonamide at 100 and 75 mg/kg/day 5 days on, 5 days off for 2 cycles, as well as 50 mg/kg/day administered daily, demonstrated dose-dependent antitumor activity in the MDA-MB 468 xenogaft model (Table 2 and FIG. 5). In combination with docetaxel at 33.3 mg/kg/day, N-[2-[(4-hydroxyphenyl)amino]-3-pyridyl]-4-methoxybenzenesulfonamide demonstrated greater than additive responses with all three doses of N-[2-[(4-hydroxyphenyl)amino]-3-pyridyl]-4-methoxybenzenesulfonamide tested.

TABLE 2
In vivo efficacy of N-[2-[(4-hydroxyphenyl)amino]-3-pyridyl]-4-
methoxybenzenesulfonamide alone and in combination with docetaxel in the MDA-MB-468
breast subcutaneous flank xenografts grown in female nude mice. Tumors were size matched
at ˜231 mm³ and therapy was initiated.

| | | | | % | |
|---|---|---|---|---|---|
| | | | Tumor | T/C^{b} | |
| | Dose | Route | Volume^{a} | Day | |
| Compound | (mg/kg/day) | Schedule | Day 38 | 38 | % ILS^{c} |
| N-[2-[(4- | 100 | p.o., q.d., (5 days on, | 638 ± 48 | 25*** | 78*** |
| hydroxyphenyl)amino]- | | 5 days off) x2 | | | |
| 3- | | | | | |
| pyridyl]-4- | | | | | |
| methoxybenzenesulfonamide | | | | | |
| | 75 | | 851 ± 55 | 33*** | 52*** |
| | 0 | | 2563 ± 183 | | |
| N-[2-[(4- | 50 | p.o., q.d x21 | 1244 ± 59 | 46*** | 41*** |
| hydroxyphenyl)amino]- | | | | | |
| 3- | | | | | |
| pyridyl]-4- | | | | | |
| methoxybenzenesulfonamide | | | | | |
| Vehicle N-[2- | 0 | | 2709 ± 123 | | |
| [(4- | | | | | |
| hydroxyphenyl)amino]- | | | | | |
| 3- | | | | | |
| pyridyl]-4- | | | | | |
| methoxybenzenesulfonamide | | | | | |
| Docetaxel | 40 | i.v., q.d x1 | 1008 ± 58 | 36*** | 52*** |
| | 33.3 | | 551 ± 31 | 19*** | 67*** |
| | 16.7 | | 1909 ± 101 | 68*** | 26*** |
| Vehicle | 0 | | 2826 ± 100 | | |
| (Docetaxel) | | | | | |
| N-[2-[(4- | 100/33.3 | p.o., q.d., (5 days on, 5 | 105 ± 13 | 4*** | 170***^{ef} |
| hydroxyphenyl)amino]- | | | | | |
| 3- | | | | | |
| pyridyl]-4- | | | | | |
| methoxybenzenesulfonamide/ | | | | | |
| Docetaxel | | | | | |
| | 75/33.3 | days off x2/i.p., q.d | 238 ± 19 | 8*** | 130***^{eg} |
| | | x1 | | | |
| | 100/16.7 | | 846 ± 58 | 29*** | 52*** |
| Vehicle (N-[2- | 0/0 | | 2891 ± 104 | | |
| [(4- | | | | | |
| hydroxyphenyl)amino]- | | | | | |
| 3- | | | | | |
| pyridyl]-4- | | | | | |
| methoxybenzenesulfonamide/ | | | | | |
| Doce) | | | | | |
| N-[2-[(4- | 50/33.3 | p.o., q.d.x21/i.p., q.d | 186 ± 13 | 7*** | 118*** |
| hydroxyphenyl)amino]- | | x1 | | | |
| 3- | | | | | |
| pyridyl]-4- | | | | | |
| methoxybenzenesulfonamide/ | | | | | |
| Docetaxel | | | | | |
| | 50/16.7 | | 786 ± 46 | 27*** | 52*** |
| Vehicle (N-[2- | | | 2699 ± 93 | | |
| [(4- | | | | | |
| hydroxyphenyl)amino]- | | | | | |
| 3- | | | | | |
| pyridyl]-4- | | | | | |
| methoxybenzenesulfonamide/ | | | | | |
| Doce) | | | | | |

^{a}Mean ± SEM
^{b}Ratio of tumor volume for treated vs. control (vehicle)
^{c}% Median increase in % ILS compared to vehicle treated controls in time to 1 cc
^{d}p values vs. vehicle, * <0.05, ** <0.01, *** <0.001
^{e}p < 0.001 compared to docetaxel 33.3 mkd
^{f}p < 0.001 compared to ABT-N-[2-[(4-hydroxyphenyl)amino]-3-pyridyl]-4-methoxybenzenesulfonamide 100 mkd, ^{g} p < 0.001 compared to N-[2-[(4-hydroxyphenyl)amino]-3-pyridyl]-4-methoxybenzenesulfonamide, 75 mkd.

Efficacy of N-[2-[(4-hydroxyphenyl)amino]-3-pyridyl]-4-methoxybenzenesulfonamide in the HT-29 Flank Xenograft Model

Tumor cells derived from serially passaged tumor fragments were inoculated s.c. in female nude mice on day 0. On day 10, mice bearing established tumors were size matched at ˜236 mm³ and placed into the following groups:

1. N-[2-[(4-hydroxyphenyl)amino]-3-pyridyl]-4-methoxybenzenesulfonamide—100, and 75 mg/kg/day (p.o., q.d., 5 days on, 5 days off ×2)

2. N-[2-[(4-hydroxyphenyl)amino]-3-pyridyl]-4-methoxybenzenesulfonamide—50 and 0 mg/kg/day (p.o., q.d. ×21 or p.o., b.i.d. ×21)

3. 5-FU—40, 30, 20 and 0 mg/kg/day (ip, qd ×1)

Combination Therapy—100/30, 75/30, mg/kg/day (p.o., q.d., 5 days on, 5 days off ×2/ip, qd ×1), 50/10 mg/kg/day (p.o., q.d., ×21/ip, qd ×1).

In the HT-29 colon xenograft model, N-[2-[(4-hydroxyphenyl)amino]-3-pyridyl]-4-methoxybenzenesulfonamide administered as a single agent at 100 and 75 mg/kg/day on a 5 days on, 5 days off schedule for 2 cycles, demonstrated significant antitumor activity. N-[2-[(4-hydroxyphenyl)amino]-3-pyridyl]-4-methoxybenzenesulfonamide at 50 mg/kg/day administered either on a q.d., or b.i.d., schedule demonstrated similar efficacy in this model confirming the observation made with the Calu-6 model. In combination with 5-FU at 30 mg/kg/day, N-[2-[(4-hydroxyphenyl)amino]-3-pyridyl]-4-methoxybenzenesulfonamide at the higher doses demonstrated additive responses.

TABLE 3
In vivo efficacy of N-[2-[(4-hydroxyphenyl)amino]-3-pyridyl]-4-
methoxybenzenesulfonamide alone and in combination with 5-FU in the HT-29 colon
subcutaneous flank xenografts grown in male nude mice. Tumors were size matched
at ˜236 mm³ and therapy was initiated.

| | | | | % | |
|---|---|---|---|---|---|
| | | | Tumor | T/C^{b} | |
| | Dose | Route | Volume^{a} | Day | |
| Compound | (mg/kg/day) | Schedule | Day 38 | 38 | % ILS^{c} |
| N-[2-[(4- | 100 | p.o., q.d., (5 days on, | 619 ± 40 | 28*** | 75*** |
| hydroxyphenyl)amino]- | | 5 days off) x2 | | | |
| 3- | | | | | |
| pyridyl]-4- | | | | | |
| methoxybenzenesulfonamide | | | | | |
| | 75 | | 942 ± 46 | 43*** | 50*** |
| | 0 | | 2210 ± 82 | | |
| N-[2-[(4- | 50 | p.o., b.i.d x21 | 1158 ± 62 | 51*** | 36*** |
| hydroxyphenyl)amino]- | | | | | |
| 3- | | | | | |
| pyridyl]-4- | | | | | |
| methoxybenzenesulfonamide | | | | | |
| Vehicle (N-[2- | 0 | | 2264 ± 78 | | |
| [(4- | | | | | |
| hydroxyphenyl)amino]- | | | | | |
| 3- | | | | | |
| pyridyl]-4- | | | | | |
| methoxybenzenesulfonamide) | | | | | |
| N-[2-[(4- | 50 | p.o., q.d x21 | 1244 ± 55 | 55*** | 36*** |
| hydroxyphenyl)amino]- | | | | | |
| 3- | | | | | |
| pyridyl]-4- | | | | | |
| methoxybenzenesulfonamide | | | | | |
| Vehicle (N-[2- | 0 | | 2248 ± 63 | | |
| [(4- | | | | | |
| hydroxyphenyl)amino]- | | | | | |
| 3- | | | | | |
| pyridyl]-4- | | | | | |
| methoxybenzenesulfonamide) | | | | | |
| 5-FU | 40 | i.p., q.d x5 | 973 ± 45 | 41*** | 50*** |
| | 30 | | 525 ± 35 | 22*** | 75*** |
| | 20 | | 1413 ± 80 | 59*** | 25*** |
| Vehicle (5-FU) | 0 | | 2399 ± 114 | | |
| N-[2-[(4- | 100/30 | p.o., q.d., (5 days on, 5 | 127 ± 14 | 5*** | 150***^{ef} |
| hydroxyphenyl)amino]- | | | | | |
| 3- | | | | | |
| pyridyl]-4- | | | | | |
| methoxybenzenesulfonamide/ | | | | | |
| 5- | | | | | |
| FU | | | | | |
| | 75/30 | days off) x2/i.p., q.d | 433 ± 38 | 17*** | 100***^{eg} |
| | | x1 | | | |
| Vehicle (N-[2- | 0/0 | | 2572 ± 125 | | |
| [(4- | | | | | |
| hydroxyphenyl)amino]- | | | | | |
| 3- | | | | | |
| pyridyl]-4- | | | | | |
| methoxybenzenesulfonamide/ | | | | | |
| 5- | | | | | |
| FU) | | | | | |
| ABT-N-[2-[(4- | 50/30 | p.o., q.d. x21/i.p., q.d | 174 ± 14 | 7*** | 86*** |
| hydroxyphenyl)amino]- | | x1 | | | |
| 3- | | | | | |
| pyridyl]-4- | | | | | |
| methoxybenzenesulfonamide/ | | | | | |
| 5- | | | | | |
| FU | | | | | |
| Vehicle (N-[2- | | | 2449 ± 95 | | |
| [(4- | | | | | |
| hydroxyphenyl)amino]- | | | | | |
| 3- | | | | | |
| pyridyl]-4- | | | | | |
| methoxybenzenesulfonamide/ | | | | | |
| 5- | | | | | |
| FU) | | | | | |

^{a}Mean ± SEM
^{b}Ratio of tumor volume for treated vs. control (vehicle)
^{c}% Median increase in % ILS compared to vehicle treated controls in time to 1 cc
^{d}p values vs. vehicle, * <0.05, ** <0.01, *** <0.001
^{e}p < 0.001 compared to 5-FU 30 mkd
^{f}p < 0.001 compared to N-[2-[(4-hydroxyphenyl)amino]-3-pyridyl]-4-methoxybenzenesulfonamide 100 mkd, ^{g} p < 0.001 compared to N-[2-[(4-hydroxyphenyl)amino]-3-pyridyl]-4-methoxybenzenesulfonamide, 75 mkd

To summarize, efficacy of N-[2-[(4-hydroxyphenyl)amino]-3-pyridyl]-4-methoxybenzenesulfonamide was tested in the Calu-6 NSCL, MDA-MB-468 breast and HT-29 colon xenograft models. As a single agent, N-[2-[(4-hydroxyphenyl)amino]-3-pyridyl]-4-methoxybenzenesulfonamide demonstrated dose-dependent efficacy in all three xenograft models. Efficacy of N-[2-[(4-hydroxyphenyl)amino]-3-pyridyl]-4-methoxybenzenesulfonamide on a q.d. vs. b.i.d. was tested in the Calu-6 NSCL and HT-29 colon xenograft models. N-[2-[(4-hydroxyphenyl)amino]-3-pyridyl]-4-methoxybenzenesulfonamide demonstrated equal efficacy when administered in either q.d. or b.i.d. for 21 days in both models.

N-[2-[(4-hydroxyphenyl)amino]-3-pyridyl]-4-methoxybenzenesulfonamide, in combination with cisplatin (Calu-6 NSCLC), docetaxel (MDA-MB-468) or 5-FU (HT-29) showed equal to or greater than additive efficacy compared to single agents alone.

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A method of administering an oral tubulin binding agent, the method comprising administering the oral tubulin binding agent at least once per day over an extended period of time.

2. The method of claim 1 wherein the oral tubulin binding agent binds to the colchicine binding site.

3. The method of claim 1 wherein the oral tubulin binding agent is N-[2-[(4-hydroxyphenyl)amino]-3-pyridyl]-4-methoxybenzenesulfonamide.

4. The method of claim 1 wherein the oral tubulin binding agent is administered once per day.

5. The method of claim 1 wherein the oral tubulin binding agent is administered twice per day.

6. The method of claim 1 wherein the oral tubulin binding agent is administered in an amount between about 25 mg and about 200 mg per day.

7. The method of claim 1 wherein the extended period of time is between about 7 and about 28 days.

8. The method of claim 1 wherein the extended period of time is about 7 days.

9. The method of claim 1 wherein the extended period of time is about 14 days.

10. The method of claim 1 wherein the extended period of time is about 21 days.

11. The method of claim 1 wherein the extended period of time is about 28 days.

12. A method of treating a disease, the method comprising administering an oral tubulin binding agent at least once per day over an extended period of time.

13. The method of claim 12 wherein the disease is cancer.

14. The method of claim 13 wherein the cancer is selected from the group consisting of leukemia, neuroblastoma, cervical, colorectal, renal, and melanoma.

15. The method of claim 14 wherein the cancer is colorectal.
